(19) United States
(12) Patent Application Publication
(10) Pub. No.: US 2008/0311062 A1
(43) Pub. Date: Dec. 18, 2008

(54) Water-In-Oil Emulsions For Hair Treatment

(76) Inventors: Kelvin Brian Dickinson, Wirral (GB); Anand Ramchandra Mahadeshwar, Hamburg (DE); Ruby Loo Bick Tan-Walker, Wirral (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/793,907

(22) PCT Filed: Nov. 7, 2005
(86) PCT No.: PCT/EP2005/011973

§ 371 Date: Jun. 10, 2008

(30) Foreign Application Priority Data
Dec. 23, 2004 (EP) 04258091.0

(51) Int. Cl. A61K 8/04; A61Q 5/00

(52) U.S. Cl. 424/70.1

(57) ABSTRACT

The present invention provides a water-in-oil emulsion for hair treatment comprising: (a) an oil phase comprising: (i) a first oily component which is one or more glyceride fatty esters, and (ii) a second oily component which is one or more hydrocarbon oils of average carbon chain length less than 20 carbon atoms; (b) a hydrophilic phase comprising: (i) water, (ii) a nonionic emulsifier which is an ethoxylated alcohol having an HLB of at least 6, and (c) dispersed particles of a hair treatment wax.

### FIELD OF THE INVENTION

This invention relates to water-in-oil emulsions for hair treatment which have enhanced sensory properties and enhanced compatibility with hair benefit agents.

### BACKGROUND OF INVENTION AND PRIOR ART

Consumers oil hair both pre wash and post wash. Pre wash oiling is done as it is believed that oils nourish hair and protect it during the wash process. Post-wash oiling is done for manageability and styling. The oiling habit is widely practised by around 800 million people across the Central Asia and Middle East region.

Coconut oil is by far the most common oil used in the Central Asia and Middle East region for hair care. It offers a high level of conditioning benefits, but with the drawback of greasy feel.

EP 1289479 discloses hair oils which incorporate a specific blend of oil types (glyceride fatty esters and hydrocarbon oils) and which can deliver an equivalent level of conditioning benefits to coconut oil, but with superior sensory properties, in particular less greasy feel.

It would be desirable to incorporate hair benefit agents such as hair treatment waxes into such oils, in order to improve the conditioning, manageability and styling behaviour of the hair after application of the product.

However a problem is that such agents are generally not compatible with the oil and cannot be incorporated into the oil in a stable manner. When such agents are combined with hair oils at effective levels, they tend to form a two-phase system, with an unattractive appearance and a tendency to separate due to differing density of the two phases.

The present inventors have found that this problem can be solved if a particular type of nonionic emulsifier is formulated with the oil. The invention provides an oil microstructure which has enhanced sensory properties and enhanced compatibility with hair benefit agents such as hair treatment waxes.

### DEFINITION OF THE INVENTION

The present invention provides a water-in-oil emulsion for hair treatment comprising:

(a) an oil phase comprising:
(i) a first oily component which is one or more glyceride fatty esters, and
(ii) a second oily component which is one or more hydrocarbon oils of average carbon chain length less than 20 carbon atoms;

(b) a hydrophilic phase comprising:
(i) water,
(ii) a nonionic emulsifier which is an ethoxylated alcohol having an HLB of at least 6, and

(c) dispersed particles of a hair treatment wax.

### DETAILED DESCRIPTION OF THE INVENTION

(a) (i) Glyceride Fatty Ester

The water-in-oil emulsion of the invention comprises an oil phase comprising a first oily component which is one or more glyceride fatty esters.

By “glyceride fatty esters” is meant the mono-, di-, and tri-esters formed between glycerol and long chain carboxylic acids such as C₆-C₃₀ carboxylic acids. The carboxylic acids may be saturated or unsaturated or contain hydrophilic groups such as hydroxyl.

Preferred glyceride fatty esters are derived from carboxylic acids of carbon chain length ranging from C₆ to C₂₄, preferably C₁₀ to C₂₂, most preferably C₁₂ to C₁₈.

Suitable glyceride fatty esters for use in emulsions of the invention will generally have a viscosity at ambient temperature (25 to 30° C.) of from 0.01 to 0.8 Pa.s , preferably from 0.015 to 0.6 Pa.s, more preferably from 0.02 to 0.065 Pa.s as measured by a Carri-Med CSL2 100 controlled stress rheometer, from TA Instruments Inc., New Castle, Del. (USA).

A variety of these types of materials are present in vegetable and animal fats and oils, such as camellia oil, coconut oil, castor oil, safflower oil, sunflower oil, peanut oil, cottonseed oil, corn oil, olive oil, cod liver oil, almond oil, avocado oil, palm oil, sesame oil, lanolin and soybean oil. These have various ranges of carbon chain lengths depending on the source, typically between about 12 to about 18 carbon atoms. Synthetic oils include trimyristin, triolein and tristearin glyceryl dilaurate. Vegetable derived glyceride fatty esters are particularly preferred, and specific examples of preferred materials for inclusion in emulsions of the invention as sources of glyceride fatty esters include almond oil, castor oil, coconut oil, sesame oil, sunflower oil and soybean oil. Coconut oil, sunflower oil, almond oil and mixtures thereof are particularly preferred.

The glyceride fatty ester may be present in emulsions of the invention as a single material or as a blend.

The total content of glyceride fatty ester in emulsions of the invention suitably ranges from 10% to 95%, preferably from 20% to 80%, by weight based on total weight of the emulsion.

(a)(ii) Hydrocarbon Oil

The oil phase of the water-in-oil emulsion of the invention comprises a second oily component which is one or more hydrocarbon oils of average carbon chain length less than 20 carbon atoms.

Suitable hydrocarbon oils include cyclic hydrocarbons, straight chain aliphatic hydrocarbons (saturated or unsaturated), and branched chain aliphatic hydrocarbons (saturated or unsaturated). Straight chain hydrocarbon oils will typically contain from about 6 to about 16 carbon atoms, preferably from about 8 up to about 14 carbon atoms. Branched chain hydrocarbon oils can and typically may contain higher numbers of carbon atoms, e.g. from about 6 up to about 20 carbon atoms, preferably from about 8 up to about 18 carbon atoms.

Suitable hydrocarbon oils will generally have a viscosity at ambient temperature (25 to 30° C.) of from 0.0001 to 0.5 Pa.s, preferably from 0.001 to 0.05 Pa.s, more preferably from 0.001 to 0.02 Pa.s as measured by a Carri-Med CSL2 100 controlled stress rheometer, from TA Instruments Inc., New Castle, Del. (USA).

A preferred hydrocarbon oil is light mineral oil. Mineral oils are clear oily liquids obtained from petroleum oil, from which waxes have been removed, and the more volatile fractions removed by distillation. The fraction distilling between 250° C. to 300° C. is termed mineral oil, and it consists of a mixture of hydrocarbons, in which the number of carbon atoms per hydrocarbon molecule generally ranges from C₁₀ to C₄₀. Mineral oil may be characterised in terms of its viscosity, where light mineral oil is relatively less viscous than heavy mineral oil, and these terms are defined more specifically in the U.S. Pharmacopoeia, 22nd revision, p. 899 (1990). A commercially available example of a suitable light mineral oil for use in the invention is Sirius M40 (carbon chain length C₁₀-C₂₈, mainly C₁₂-C₂₀, viscosity 4.3×10⁻³ Pa.s), available from Silkolene.

Other hydrocarbon oils that may be used in the invention include relatively lower molecular weight hydrocarbons including linear saturated hydrocarbons such a tetradecane, hexadecane, and octadecane, cyclic hydrocarbons such as dioctylcyclohexane (e.g. CETIOL S from Henkel), branched chain hydrocarbons (e.g. ISOPAR L and ISOPAR V from Exxon Corp.).

The hydrocarbon oil may be present in emulsions of the invention as a single material or as a blend.

The total content of hydrocarbon oil in emulsions of the invention suitably ranges from 5% to 90%, preferably from 20% to 80%, by weight based on total weight of the emulsion.

The glyceride fatty ester:hydrocarbon oil weight ratio in emulsions of the invention may suitably range from 90:10 to 10:90, preferably from 80:20 to 20:80, more preferably from 60:40 to 40:60. Particularly preferred are blends of [coconut oil and/or sunflower oil and/or almond oil] and light mineral oil, in which the [coconut oil and/or sunflower oil and/or almond oil]:light mineral oil weight ratio is about 50:50.

(b)(i) Water

The hydrophilic phase of the water-in-oil emulsion of the invention comprises water, suitably at a level of from about 2% by weight based on total weight of the emulsion. Suitably the water level does not exceed about 10% by weight based on total weight of the emulsion. Preferably the water level ranges from 3 to 7%, more preferably from 4 to 6% by weight based on total weight of the emulsion.

(b)(ii) Nonionic Emulsifier

The water-in-oil emulsion of the invention comprises a nonionic emulsifier which is an ethoxylated alcohol having an HLB of at least 6.

Suitable ethoxylated alcohols are commercially available and include the primary aliphatic alcohol ethoxylates and secondary aliphatic alcohol ethoxylates. The length of the polyethenoxy chain can be adjusted to achieve the desired balance between the hydrophobic and hydrophilic elements.

The HLB value of the ethoxylated alcohol suitably ranges from 6 to 12, preferably from 7 to 10, more preferably from 7 to 9.

Examples of suitable ethoxylated alcohols include the condensation products of a higher alcohol (e.g., an alkanol containing about 8 to 16 carbon atoms in a straight or branched chain configuration) condensed with about 2.5 to 20 moles of ethylene oxide.

A preferred group of the foregoing ethoxylated alcohols are the Neodol ethoxylates (Shell Co.), which are higher aliphatic, primary alcohols containing about 9 to 15 carbon atoms condensed with about 2.5 to 20 moles of ethylene oxide. Specific examples are C9 to 11 alkanol condensed with 2.5 to 10 moles of ethylene oxide (Neodol 91-8 or Neodol 91-5), C12 to 13 alkanol condensed with 3 moles ethylene oxide (Neodol 23-3), C12 to 15 alkanol condensed with 12 moles ethylene oxide (Neodol 25-12), C14 to 15 alkanol condensed with 13 moles ethylene oxide (Neodol 45-13), and the like. Such ethoxylates have an HLB (hydrophobic lipophilic balance) value of about 7 to 10. Most preferred is Neodol 23-3, with an HLB of about 8.

The level of nonionic emulsifier in emulsions of the invention suitably ranges from 10 to 40%, preferably from 15 to 35%, by weight based on total weight of the emulsion.

(c) Hair Treatment Wax

The water-in-oil emulsion of the invention comprises dispersed particles of a hair treatment wax.

Suitable waxes are hydrophobic materials with a water solubility of less than 0.01 g/l at room temperature (25° C.).

Suitable waxes have a melting point in the range of from 30° C. to 150° C., preferably from 40° C. to 100° C., more preferably from 45° C. to 80° C.

Suitable waxes may be selected from naturally occurring waxes, synthetic hydrocarbon waxes, synthetic silicone waxes and mixtures thereof.

Naturally occurring waxes may be obtained directly or indirectly from natural plant, animal or mineral sources, for example waxes obtained by the hydrogenation of vegetable oil, animal fats and oils and natural waxes from plants.

Waxes may contain substantially one chemical compound or a mixture of chemical compounds and can be used singly or as a mixture of two or more different waxes.

Specific examples of suitable waxes are those based on hydrogenated vegetable oil, candelilla wax (extracted from the candelilla plant) carnauba wax (extracted from the palm tree carnauba), silicone-urethane copolymers, cotton wax, bayberry wax, Chinese wax, spermaceti, montan wax, rice bran wax, lanolin, kapok wax, lanolin acetate or other lanolin esters and/or ethers, sugar cane wax, hexyl laurate, jojoba wax, shellac, paraffin wax, cholesterol, hydrogenated castor oil, petrolatum, microcrystalline wax, cocoa butter and the like, optionally hydrogenated where this is not already specified and where this is appropriate in order to increase the melting point of the wax so that it lies in a suitable range as described above.

Preferred examples of suitable waxes are microcrystalline wax, beeswax and silicone-urethane copolymers.

By the term “microcrystalline wax” is generally meant a refined mixture of solid, saturated hydrocarbons, mainly branched paraffin, obtained from petroleum.

By the term “beeswax” it is intended to include natural beeswax obtained from honeycombs in either an unpurified or preferably purified state and synthetic and substitute beeswax, as well as beeswax that has been modified, for example organically modified. The term “synthetic and substitute” beeswax includes substances that have similar properties to those of natural beeswax.

Suitable organically modified beeswaxes include, for example, PEG-8 beeswax, polyglycerol-3 beeswax and siliconized beeswax such as dimethicone copolyol beeswax.

A suitable example of a silicone-urethane copolymer is a polydimethylsiloxane-polypropylene glycol ether/isophorone diisocyanate (IPDI) copolymer. Such a material is marketed under the name Polyderm PPI-SI-WI by Alzo Inc.

Mixtures of any of the above described hair treatment waxes may also be used.

The total amount of hair treatment wax in emulsions of the invention suitably ranges from 0.5 to 7%, preferably from 1.5 to 6%, by weight based on total weight of the emulsion.

Process

A preferred process for preparing a water-in-oil emulsion according to the present invention comprises the following steps:

(I) blending the oil phase [(a)] and the hair treatment wax [(c)];

(II) heating the blend so obtained to melt the wax, and

(III) mixing the blend with the nonionic emulsifier [(b)(ii)] and the water [(b)(i)].

Product Form and Usage

Compositions of this invention are preferably for application directly to the hair in neat form, either before or after shampooing.

Accordingly the invention also provides a method of treating hair comprising the step of applying a water-in-oil emulsion as described above directly to the hair as a pre-wash treatment or as a post-wash treatment.

Optional Ingredients

Compositions of this invention may contain any other ingredient normally used in hair treatment formulations. These other ingredients may include preservatives such as phenoxetol® (2-phenoxyethanol), colouring agents, antioxidants such as BHT (butylhydroxytoluene), fragrances and antimicrobials such as Glycacil-L ® (iodopropynyl butylcarbamate). Each of these ingredients will be present in an amount effective to accomplish its purpose. Generally these optional ingredients are included individually at a level of up to about 5% by weight based on total weight of the emulsion.

The invention is further illustrated by way of the following Examples, in which all percentages are by weight based on total weight unless otherwise stated.

### EXAMPLES

Water-in-oil emulsions containing hair treatment waxes were prepared, having ingredients as shown in the following Table:

| | Formulation Examples: | | |
|---|---|---|---|
| Ingredient | Example 1 | Example 2 | Example 3 |
| Sunflower oil | 31.85 | 30.35 | 32.00 |
| Light mineral oil | 31.85 | 30.35 | 32.00 |
| (Sirius M40, from | | | |
| Silkolene) | | | |
| Nonionic emulsifier | 30.00 | 30.00 | 30.00 |
| (NEODOL 23-3, from | | | |
| Shell Co.) | | | |
| Water | to 100 | to 100 | to 100 |
| Microcrystalline wax | 2.00 | — | — |
| (MULTIWAX W-445, from | | | |
| Crompton Witco) | | | |
| Beeswax | — | 5.00 | — |
| Silicone polyurethane | — | — | 1.00 |
| wax (POLYDERM PPI-SI- | | | |
| WI, from Alzo Inc.) | | | |

In each case, the sunflower oil and light mineral oil were blended with the wax ingredient, and the blend so obtained was heated to 65 degrees C. to melt the wax. The NEODOL 23-3 was then added, followed by the water. The resulting formulations were milky water-in-oil emulsions.

Comparative evaluations of the above formulations according to the invention were carried out using a control formulation of 50 wt % Sirius M40 and 50 wt % sunflower oil.

The formulations of Examples 1 to 3 were each compared against the control formulation across a number of performance attributes. Evaluation was carried out in two stages:

(i) Post Oiling.

Half of the hair of a mannequin head was oiled with the control formulation and the other half with the test formulation (Example 1, 2 or 3 respectively). 2.0 ml of formulation was used to oil the individual half head. After one hour the mannequin head was assessed by an expert salon hairdresser.

(ii) Post Wash.

3.5 ml of a commercial shampoo was measured and applied onto the oiled half head, followed by washing and rinsing in accordance with normal procedures. The shampooing and rinsing procedure was repeated for a second application. The same procedure was followed for the other oiled half head. After washing and rinsing was complete the mannequin head was allowed to dry at normal temperature (20 to 25 degrees C.). On drying the mannequin head was assessed by an expert salon hairdresser.

The following results were obtained:

Post oiling:

Compared to the control, the formulation of Example 1 gave significantly (>99%) better hair body and significantly (>90%) better hair conditioning. The formulation of Example 1 was also found to have significantly (>90%) reduced product sticky feel compared to the control.

Compared to the control, the formulation of Example 2 gave significantly (>95%) better hair body and significantly (>90%) better hair conditioning. The formulation of Example 2 was also found to have significantly (>90%) reduced product sticky feel compared to the control.

Compared to the control, the formulation of Example 3 gave significantly (>90%) better hair conditioning and significantly (>90%) reduced hair sticky feel. The formulation of Example 3 was also found to have significantly (>90%) reduced product sticky feel compared to the control.

Post wash:

Compared to the control, the formulation of Example 1 gave significantly (>90%) better hair conditioning and significantly (>95%) better hair shine.

Compared to the control, the formulation of Example 2 gave significantly (>95%) better hair conditioning.

Compared to the control, the formulation of Example 3 gave significantly (>90%) better hair body, significantly (>90%) better hair conditioning and significantly (>90%) better hair shine.

In a further test, attempts to disperse the wax ingredients of Examples 1, 2 and 3 directly into the control formulation resulted in unstable products which separated immediately. By contrast, the formulations of Examples 1, 2 and 3 remained stable on storage for over 6 months at 25 degrees C.

1. A water-in-oil emulsion for hair treatment comprising:
(a) an oil phase comprising:
(i) a first oily component which is one or more glyceride fatty esters, and
(ii) a second oily component which is one or more hydrocarbon oils of average carbon chain length less than 20 carbon atoms;
(b) a hydrophilic phase comprising:
(i) water,
(ii) a nonionic emulsifier which is an ethoxylated alcohol having an HLB of at least 6, and
(c) dispersed particles of a hair treatment wax.

2. An emulsion according to claim 1, in which the source of glyceride fatty esters is selected from coconut oil, sunflower oil, almond oil and mixtures thereof.

3. An emulsion according to claim 1, in which the total content of glyceride fatty ester ranges from 20% to 80% by weight based on total weight of the emulsion.

4. An emulsion according to claim 1, in which the hydrocarbon oil is light mineral oil.

5. An emulsion according to claim 1, in which the total content of hydrocarbon oil ranges from 20% to 80% by weight based on total weight of the emulsion.

6. An emulsion according to claim 1, in which the glyceride fatty ester:hydrocarbon oil weight ratio ranges from 95:5 to 5:95, preferably from 90:10 to 10:90, most preferably from 80:20 to 20:80.

7. An emulsion according to claim 1, in which the water level ranges from 3 to 7%, more preferably from 4 to 6% by weight based on total weight of the emulsion.

8. An emulsion according to claim 1, in which the HLB value of the ethoxylated alcohol ranges from 6 to 12, preferably from 7 to 10, more preferably from 7 to 9.

9. An emulsion according to claim 8, in which the ethoxylated alcohol is a higher aliphatic, primary alcohol containing about 9 to 15 carbon atoms, condensed with about 2.5 to 10 moles of ethylene oxide.

10. An emulsion according to claim 9, in which the ethoxylated alcohol is C12 to 13 alkanol condensed with 3 moles ethylene oxide.

11. An emulsion according to claim 1, in which the hair treatment wax is selected from naturally occurring waxes, synthetic hydrocarbon waxes, synthetic silicone waxes and mixtures thereof.

12. An emulsion according to claim 11, in which the hair treatment wax is microcrystalline wax.

13. An emulsion according to claim 11, in which the hair treatment wax is beeswax.

14. An emulsion according to claim 11, in which the hair treatment wax is a silicone-urethane copolymer.

15. A method of treating hair comprising the step of applying a water-in-oil emulsion according to claim 1 directly to the hair as a pre-wash treatment or as a post-wash treatment.
